(19) United States
(12) Patent Application Publication
(10) Pub. No.: US 2004/0166575 A1
(43) Pub. Date: Aug. 26, 2004

(54) Inosine producing bacterium belonging to the genus Bacillus and method for producing inosine

(76) Inventors: Misa Tominaga, Saga (JP); Keishi Shimazaki, Saga (JP); Kiyoshi Matsuno, Kawasaki-shi (JP); Minoru Yamashita, Kawasaki-shi (JP)

Correspondence Address: AJINOMOTO CORPORATE SERVICES, LLC, INTELLECTUAL PROPERTY DEPARTMENT, 1120 CONNECTICUT AVE., N.W., WASHINGTON, DC, 20036, US

(21) Appl. No.: 10/772,272

(22) Filed: Feb. 6, 2004

(30) Foreign Application Priority Data
Feb. 17, 2003 (JP) 2003-37760

(51) Int. Cl. C12N 1/21

(52) U.S. Cl. 435/252.31

(57) ABSTRACT

Strains showing favorable growth in a medium containing 6-ethoxypurine are selected from a population of Bacillus bacteria, and a strain showing high inosine-producing ability is selected from the obtained strains to obtain a Bacillus bacterium having improved inosine-producing ability.

### BACKGROUND OF THE INVENTION

1. Field of the Invention

The present invention relates to a method for producing inosine, which is an important substance useful as a synthetic raw material to produce 5′-inosinic acid, and a novel microorganism useful for the production thereof.

2. Description of the Related Art

There are known methods for fermentative production of inosine utilizing microorganisms of the genus Bacillus, which are adenine-auxotrophic strains or adenineauxotrophic strains further imparted with resistance to various substances, including purine analogues (Japanese Patent Publication (KOKOKU) Nos. 38-23099, 54-17033, 55-2956, 55-45199, 57-14160, 57-41915 and Japanese Patent Laid-open (KOKAI) No. 59-42895), microorganisms of the genus Brevibacterium (Japanese Patent Publication (KOKOKU) Nos. 51-5075, 58-17592, Agric. Biol. Chem., 42, 399 (1978)), and so forth.

In order to obtain such mutant strains, a method of performing a mutagensis treatment such as ultraviolet irradiation or nitrosoguanidine (N-methyl-N′-nitro-N-nitrosoguanidine) treatment and screeninga target mutant strain using a suitable selection medium has been conventionally used. On the other hand, breeding of nucleic acid-producing strains using genetic engineering techniques has also been performed for microorganisms of the genus Bacillus (Japanese Patent Laid-open (KOKAI) Nos. 58-158197, 58-175493, 59-28470, 60-156388, 1-27477, 1-174385, 3-58787, 3-164185, 5-84067, 5-192164, 11-346778 (U.S. Pat. No. 6,284,495)) and microorganisms of the genus Brevibacterium (Japanese Patent Laid-open (KOKAI) No. 63-248394). Specifically, for example, a method of using a Bacillus bacterium in which the repressor protein gene (purR) of the purine operon was disrupted to efficiently produce nucleic acid compounds such as hypoxanthine, uracil, guanine and adenine has been disclosed (Japanese Patent Laid-open (KOKAI) No. 11-346778 (U.S. Pat. No. 6,284,495)).

Moreover, for Bacillus subtilis, it is known that the aforementioned repressor protein controls expressions of the purA gene involved in the biosynthesis of adenine and genes of the pyrimidine operon involved in pyrimidine biosynthesis, in addition to the genes of the purine operon (H. Zalkin et al., J. Bacteriol., 179, 7394-7402, 1997).

Furthermore, for Escherichia coli, it is known that its purine nucleoside-producing ability is improved by disrupting the succinyl-AMP synthase gene (purA) to impart adenine auxotrophy (International Patent Publication WO99/03988). Furthermore, it is also known that the purine nucleoside-producing ability is improved by disrupting the purine nucleoside phosphorylase gene (deoD) to repress the degradation of inosine and guanosine into hypoxanthine and guanine (International Patent Publication WO99/03988).

### SUMMARY OF THE INVENTION

An object of the present invention is to create a microorganism suitable for the production of inosine by fermentation and provide a method for producing inosine using such a microorganism.

It is a further object of the present invention to provide a Bacillus bacterium which is modified so that growth inhibition by 6-ethoxypurine is reduced and has inosine-producing ability.

It is a further object of the present invention to provide the Bacillus bacterium as described above, which is a mutant strain derived from a parent strain belonging to the genus Bacillus and shows favorable growth as compared with the parent strain when cultured in a medium containing 6-ethoxypurine.

It is a further object of the present invention to provide the bacterium as described above, wherein the medium has an ethoxypurine content of 2000 mg/L.

It is a further object of the present invention to provide the bacterium as described above, wherein the medium is a solid medium.

It is a further object of the present invention to provide the bacterium as described above, wherein when the bacterium is cultured by inoculationg a suspension of the bacterium to a solid medium containing 6-ethoxypurine and a solid medium not containing 6-ethoxypurine, the bacterium shows a relative growth degree of 80 or more, which is defined by the following equation:

Relative growth degree (%)=[colony diameter (mm) observed in the medium containing 6-ethoxypurine]/[colony diameter (mm) observed in the medium not containing 6-ethoxypurine]×100

It is a further object of the present invention to provide the bacterium as described above, wherein the solid medium containing 6-ethoxypurine has a 6-ethoxypurine content of 2000 mg/L.

It is a further object of the present invention to provide the bacterium as described above, wherein the solid medium is a minimal medium.

It is a further object of the present invention to provide the bacterium as described above, which is deficient in one or more genes negatively acting on the biosynthesis of inosine or involved in degradation of inosine, and selected from a purine operon repressor gene, succinyl-AMP synthase gene and purine nucleoside phosphorylase gene.

It is a still further object of the present invention to provide a method for producing a Bacillus bacterium having improved inosine-producing ability, which comprises selecting strains showing favorable growth in a medium containing 6-ethoxypurine from a population of Bacillus bacteria and selecting a strain showing high inosine-producing ability from the obtained strains.

It is even a further object of the present invention to provide the method as described above, wherein the population of Bacillus bacteria is obtained by subjecting a parent strain belonging to the genus Bacillus to a mutagenesis treatment.

According to the present invention, a microorganism having an inosine-producing ability and a method for producing inosine using the microorganism are provided.

### BRIEF DESCRIPTION OF THE DRAWINGS

FIG. 1 is a graph showing inosine (HxR) and hypoxanthine (Hyp) production amounts of a 6-ethoxypurine-resistant strain.

### DESCRIPTION OF THE PREFERRED EMBODIMENTS

The inventors of the present invention conducted research in order to achieve the aforementioned objects. As a result, it was found that inosine-producing ability of a Bacillus bacterium could be improved by modifying the Bacillus bacterium so that the growth inhibition by 6-ethoxypurine is reduced, and the inventors thus accomplished the present invention.

The Bacillus bacterium of the present invention is modified so that the growth inhibition by 6-ethoxypurine is reduced, and it has an inosine-producing ability.

The expression “the inosine-producing ability” as used herein means an ability of the Bacillus bacterium of the present invention to cause accumulation of inosine in a medium in an amount sufficient to enable collection of the inosine from the medium. For example, 4 g/L or more of inosine and hypoxanthine in terms of inosine amount, when the bacterium is cultured in the medium.

Examples of the Bacillus bacterium include, but are not limited to, Bacillus subtilis, Bacillus amyloliquefaciens, Bacillus pumilus and so forth.

Examples of the Bacillus subtilis include, but are not limited to, the Bacillus subtilis 168 Marburg strain (ATCC 6051), Bacillus subtilis PY79 (Plasmid, 1984, 12, 1-9) and so forth, and examples of Bacillus amyloliquefaciens include, but are not limited to, Bacillus amyloliquefaciens T (ATCC 23842), Bacillus amyloliquefaciens N (ATCC 23845) and so forth.

The Bacillus bacterium of the present invention can be obtained by modifying a strain having an inosine-producing ability so that the growth inhibition by 6-ethoxypurine is reduced. The Bacillus bacterium of the present invention can also be obtained by modifying a strain modified so that the growth inhibition by 6-ethoxypurine is reduced so as to be imparted with an inosine-producing ability or so that its inosine-producing ability is improved.

A Bacillus bacterium having an inosine-producing ability can be obtained by, for example, imparting adenine auxotrophy or, in addition to the adenine auxotrophy, resistance to a drug such as a purine analogue to a Bacillus bacterium (Japanese Patent Publication Nos. 38-23099, 54-17033, 55-2956, 55-45199, 57-14160, 57-41915 and Japanese Patent Laid-open Publication No. 59-42895). A Bacillus bacterium having an auxotrophy or drug resistance as described above can be obtained by ultraviolet irradiation or a treatment with a mutagenizing agent used for typical mutagenesis treatment such as N-methyl-N′-nitro-N-nitrosoguanidine (NTG) or EMS (ethyl methanesulfonate).

Methods for breeding a Bacillus bacterium having an inosine-producing ability include the following examples. For example, the methods encompass increasing intracellular activity of an enzyme involved in the inosine biosynthesis, specifically, increasing expression of the gene of the aforementioned enzyme. The phrase “increasing intracellular activity” means increasing the activity to a level higher than that of a non-modified Bacillus bacterium, such as a wild-type Bacillus bacterium. Examples include, but are not limited to, increasing the number of enzyme molecules per cell, increasing specific activity per enzyme molecule and so forth.

Examples of the enzyme involved in the inosine biosynthesis include, for example, phosphoribosyl pyrophosphate (PRPP) amidotransferase, phosphoribosyl pyrophosphate (PRPP) synthetase, adenosine deaminase and so forth.

Moreover, the examples also include canceling regulation of an enzyme involved in inosine biosynthesis, specifically, a method of canceling feedback inhibition of such an enzyme (WO99/03988). Examples of the means for canceling regulation of such an enzyme as mentioned above involved in the inosine biosynthesis include, for example, deletion of the purine repressor (U.S. Pat. No. 6,284,495). Examples of the method of deleting the purine repressor include disrupting a gene coding for the purine repressor (purR, GenBank Accession No. Z99104).

Moreover, the inosine-producing ability can also be increased by blocking a reaction branching off from the inosine biosynthesis and resulting in another metabolic product (WO99/03988). Examples of the reaction branching off from the inosine biosynthesis and resulting in another metabolic product include reactions catalyzed by, for example, succinyl-adenosine monophosphate (AMP) synthase, inosine-guanosine kinase, 6-phosphogluconate dehydrase, phosphoglucoisomerase and so forth. The succinyl-adenosine monophosphate (AMP) synthase is encoded by purA (GenBank Accession No. Z99104).

Furthermore, the inosine-producing ability can also be enhanced by weakening incorporation of inosine into cells. The incorporation of inosine into cells can be weakened by blocking a reaction involved in the incorporation of inosine into cells. Examples of the aforementioned reaction involved in the incorporation of the inosine into cells include a reaction catalyzed by, for example, nucleoside permease.

Furthermore, the inosine-producing ability can also be improved by reducing or eliminating inosine degradation activity (WO99/03988). Examples of the method of reducing or eliminating inosine degradation activity include a method of disrupting a gene coding for purine nucleoside phosphorylase (deoD).

Increasing the activity of a target enzyme in cells of a Bacillus bacterium can be attained by enhancing the expression of a gene coding for the enzyme. Enhancing the expression of the gene can be attained by increasing the copy number of the gene. For example, the gene fragment coding for the aforementioned enzyme can be ligated to a vector which functions in Bacillus bacteria, preferably a multi-copy type vector, to prepare a recombinant DNA, which is then used to transform the host Bacillus bacterium.

As the gene to be introduced, any of genes derived from Bacillus bacteria and genes derived from other organisms such as Escherichia bacteria may be used, so long as the gene functions in the Bacillus bacterium.

The target gene can be obtained by, for example, PCR method (polymerase chain reaction, see White, T. J. et al., Trends Genet., 5, 185 (1989)) using a chromosomal DNA of a Bacillus bacterium as a template. The chromosomal DNA can be prepared from a bacterium serving as a DNA donor by, for example, the method of Saito and Miura (see H. Saito and K. Miura, Biochem. Biophys. Acta, 72, 619 (1963), Text for Bioengineering Experiments, Edited by the Society for Bioscience and Bioengineering, Japan, pp.97-98, Baifukan, 1992) or the like. The primers for PCR can be prepared based on a known gene sequence of a Bacillus bacterium or based on information on a region conserved among genes of other bacteria of which sequences are known, or the like.

Examples of an autonomously replicable vector for introducing a target gene into a Bacillus bacterium include, for example, pUB 110, pC 194, pE 194 and so forth. Furthermore, examples of a vector for incorporating a target gene into a chromosomal DNA include vectors for E. coli, such as pHSG398 (Takara Shuzo) and pBluescript SK-(Stratagene).

To prepare a recombinant DNA, a target gene can be ligated with a vector which functions in a Bacillus bacterium by digesting the vector with a restriction enzyme suitable for the end of the target gene. The ligation is typically performed by using a ligase such as T4 DNA ligase.

To introduce the recombinant DNA prepared as described above into a Bacillus bacterium, any known method for transformation can be employed. Examples include, for instance, preparing competent cells from cells which are at the growth phase followed by introducing the DNA thereinto, (Dubunau and Davidoff-Abelson, J. Mol. Biol., 56, 209 (1971); Duncan, C. H., Wilson, G. A. and Young, F. E., Gene, 1, 153 (1977)) and a method of making host cells into protoplasts or spheroplasts, which can easily incorporate recombinant DNA, followed by introducing the recombinant DNA into the DNA-acceptor cells (Chang, S. and Choen, S. N., Molec. Gen. Genet., 168, 111 (1979)).

Copy number of a target gene can also be increased by allowing multiple copies of the gene to exist on chromosomal DNA of a Bacillus bacterium. Multiple copies of the target gene may be introduced into chromosomal DNA of a Bacillus bacterium by, for example, homologous recombination. This can be performed by targeting a sequence present on chromosomal DNA in multiple copy number. A repetitive DNA or an inverted repeat present at the end of a transposable element can be used as the sequence present on the chromosomal DNA in a multiple copy number.

Besides the above gene amplification methods, the activity of a target enzyme can be amplified by replacing an expression regulatory sequence, such as the promoter of the target gene on a chromosomal DNA or plasmid, with a stronger one. Moreover, it is also possible to introduce nucleotide substitution for several nucleotides into a promoter region of the target gene so that it is modified into a stronger one. Such modification of expression regulatory sequence may be combined with the increase of copy number of the target gene.

The substitution of an expression regulatory sequence can be performed, for example, in the same manner as that for the gene substitution described below.

Examples of the method for reducing an activity of a target enzyme of a Bacillus bacterium include treating the Bacillus bacterium with ultraviolet ray irradiation or a mutagenizing agent used in a typical mutagenesis treatment such as N-methyl-N′-nitro-N-nitrosoguanidine (NTG) or EMS (ethyl methanesulfonate) and selecting a mutant strain showing reduced activity of the target enzyme. Furthermore, a Bacillus bacterium having a reduced activity of a target enzyme can also obtained by replacing a gene coding for the target enzyme on a chromosome with a corresponding gene that does not normally function (hereinafter, also referred to as “disrupted-type gene”) by homologous recombination utilizing a gene recombination technique (Experiments in Molecular Genetics, Cold Spring Harbor Laboratory Press (1972); Matsuyama, S. & Mizushima, S., J. Bacteriol., 162, 1196 (1985)).

Substitution of a disrupted-type gene for a normal gene on a host chromosome can be performed as follows. In the following example, disruption of the purR gene is exemplifying. However, disruption of other genes, for example, purA and deoD, can also be similarly performed.

Homologous recombination is a phenomenon that when a plasmid, or the like, containing a sequence homologous to a chromosomal sequence is introduced into a cell, recombination results at the site in the sequence having homology at a certain frequency, and the introduced plasmid as a whole is incorporated into the chromosome. Thereafter, if further recombination results at the site in the sequence having homology on the chromosome, the plasmid is eliminated again from the chromosome. At this time, the gene introduced with a mutation may be fixed on the chromosome, and the native gene may be eliminated along with the plasmid, depending on the site where the recombination occurs.

By selecting such a strain, a strain in which the deletion type purR gene substitutes for the normal purR gene on the chromosome can be obtained.

Gene disruption by homologous recombination are known, as are methods using linear DNA, methods using a plasmid, including a temperature sensitive replication control region, and so forth. Furthermore, the purR gene can be disrupted by using a plasmid which contains the purR gene inserted with a marker gene, such as a drug resistance gene, and cannot replicate in a target microbial cell. That is, a transformant which has been transformed with such a plasmid as mentioned above and thus acquired the drug resistance is incorporated with the marker gene in the chromosomal DNA. Since it is highly probably that the marker gene is incorporated by the homologous recombination of the purR gene sequence at the both ends thereof, and the purR gene on a chromosome, a gene-disrupted strain can be efficiently selected.

The disrupted-type purR gene used for the gene disruption can be obtained by, specifically, using deletion of a certain region of the purR gene using digestion with a restriction enzyme and re-ligation, insertion of another DNA fragment (marker gene etc.) into the purR gene, site-directed mutagenesis (Kramer, W. and Frits, H. J., Methods in Enzymology, 154, 350 (1987)), or a treatment with a chemical agent such as sodium hyposulfite and hydroxylamine (Shortle, D. and Nathans, D., Proc. Natl. Acad. Sci. U.S.A., 75, 270 (1978)) to cause substitution, deletion, insertion, addition or inversion of one or more nucleotides in the nucleotide sequence of the coding region, promoter region or the like of the purR gene and thereby reduce or eliminate the activity of the encoded repressor, or reduce or eliminate the transcription of the purR gene. Among these methods, the method of deleting a certain region of the purR gene by digestion with a restriction enzyme and re-ligation or the method of inserting another DNA fragment into the purR gene is preferred in view of the reliability and stability.

The purR gene can be obtained from a chromosomal DNA of a microorganism having the purine operon by PCR using oligonucleotides prepared based on the known nucleotide sequence of the purR gene as primers. The purR gene can also be obtained from a chromosomal DNA library of a microorganism having the purine operon by the hybridization method using an oligonucleotide prepared based on the known nucleotide sequence of the purR gene as a probe. The nucleotide sequence of the purR gene was reported for the Bacillus subtilis 168 Marburg strain (GenBank accession No. D26185 (the coding region corresponds to the nucleotide numbers 118041 to 118898), DDBJ Accession No.Z99104 (the coding region corresponds to the nucleotide numbers 54439 to 55296)). The nucleotide sequence of the purR gene and the amino acid sequence encoded by the gene are shown in SEQ ID NOS: 11 and 12.

The primers used for PCR may be any of those which allow for amplification of the purR gene, and specific examples include oligonucleotides having the nucleotide sequences shown in SEQ ID NOS: 1 and 2.

Similarly, examples of the primers for amplification of the purA gene include oligonucleotides having the nucleotide sequences shown in SEQ ID NO: 3 and 4, and examples of the primers for amplification of the deoD gene include oligonucleotides having the nucleotide sequences shown in SEQ ID NOS: 5 to 8. The nucleotide sequences of the purA gene and deoD gene were reported as DDBJ Accession No. Z99104.

The nucleotide sequence of the purA gene and the amino acid sequence encoded by the gene are shown in SEQ ID NOS: 13 and 14. Furthermore, the nucleotide sequence of the deoD gene and the amino acid sequence encoded by the gene are shown in SEQ ID NOS: 15 and 16.

Each of the purR, purA and deoD genes used in the present invention is not necessarily required to have the full length for use in the preparation of a disrupted-type gene thereof, and it may have a length required for causing gene disruption. Furthermore, the microorganism used for obtaining each gene is not particularly limited so long as the gene has homology in such a degree that the gene causes homologous recombination with the homologous gene of the microorganism used for the creation of a gene-disrupted strain. However, it is usually preferable to use a gene derived from the same bacterium as the target Bacillus bacterium.

Examples of DNA that may cause homologous recombination with the purR, purA or deoD gene of a Bacillus bacterium include DNAs coding for an amino acid sequence represented in SEQ ID NO: 12, 14 or 16 including substitution, deletion, insertion or addition of one or several amino acid residues. The number of “several” amino acid residues mentioned above is, for example, between 2 to 50, preferably between 2 to 30, more preferably between 2 to 10.

Specific examples of DNA that can cause homologous recombination with the aforementioned purR, purA or deoD gene of Bacillus bacterium include DNAs having homology of, for example, 70% or more, preferably 80% or more, more preferably 90% or more, most preferably 95% or more, to the nucleotide sequence shown in SEQ ID NO: 11, 13 or 15. More specifically, the examples include DNAs hybridyzable with the nucleotide sequence shown in SEQ ID NO: 11, 13 or 15 under stringent conditions. Examples of the stringent conditions include a condition in which washing is performed at a salt concentration of 1×SSC, 0.1% SDS, preferably 0.1×SSC, 0.1% SDS, at 60° C.

Examples of the marker gene mentioned above include drug resistance genes such as a spectinomycinresistance gene and kanamycin-resistance gene. The spectinomycin-resistance gene of Enterococcus faecalis can be obtained by preparing the plasmid pDG1726 from the Escherichia coli ECE101 strain commercially available from the Bacillus Genetic Stock Center (BGSC) and excising the resistance gene as a cassette from the plasmid. Furthermore, the erythromycin-resistance gene of Staphylococcus aureus can be obtained by preparing the plasmid pDG646 from the Escherichia coli ECE91 strain commercially available from the Bacillus Genetic Stock Center (BGSC) and excising the resistance gene as a cassette from the plasmid. Furthermore, the kanamycinresistance gene can be obtained by performing PCR using the pDG783 plasmid containing the kanamycin-resistance gene derived from Streptococcus faecalis (which can be prepared from the Escherichia coli ECE94 strain commercially available from the Bacillus Genetic Stock Center) as a template and the primers shown in SEQ ID NOS: 9 and 10.

When a drug resistance gene is used as the marker gene, a purR gene-disrupted strain can be obtained by inserting the drug resistance gene into the purR gene at an appropriate site, transforming a microorganism with the obtained plasmid and selecting a transformant that has become drug resistant. Disruption of the purR gene on a chromosome can be confirmed by analyzing the purR gene or the marker gene on the chromosome using Southern blotting or PCR. Incorporation of the aforementioned spectinomycin-resistance gene, erythromycin-resistance gene or kanamycin-resistance gene into a chromosomal DNA can be confirmed by PCR using primers which can amplify these genes.

Hereinafter, reduction of growth inhibition in Bacillus bacterium by 6-ethoxypurine will be explained. If 6-ethoxypurine exists in a medium, growth of Bacillus bacteria is inhibited. That is, if a Bacillus bacterium is cultured in a medium containing 6-ethoxypurine, the bacterium would not grow or the growth becomes slower compared to when the bacterium is cultured in a medium not containing 6-ethoxypurine. Specifically, when the bacterium is cultured on a solid medium for a certain period of time, the diameter of colonies, for example, becomes smaller in a medium containing 6-ethoxypurine compared with that observed in a medium not containing 6-ethoxypurine. On the other hand, the Bacillus bacterium of the present invention is modified so that the aforementioned growth inhibition by 6-ethoxypurine is reduced.

In a Bacillus bacterium modified so that the growth inhibition by 6-ethoxypurine is reduced as described above, the inosine-producing ability is improved as compared to a non-modified strain. Therefore, the characteristic concerning 6-ethoxypurine can be applied to breeding of inosine producing bacteria of Bacillus bacteria. That is, a Bacillus bacterium having an improved inosine-producing ability can be produced by selecting strains exhibiting favorable growth in a medium containing 6-ethoxypurine from a population of Bacillus bacteria and selecting a strain showing high inosine-producing ability from the obtained strains.

The Bacillus bacterium of the present invention can be obtained as a mutant strain derived from a Bacillus bacterium as a parent strain. The mutagenesis treatment for obtaining such a mutant strain is not particularly limited, and examples thereof include, but are not limited to, a treatment with UV irradiation or a treatment with a mutagenizing agent used for typical mutagenesis treatment such as N-methyl-N′-nitro-N-nitrosoguanidine (NTG) and nitrous acid.

A mutant strain whereby growth inhibition by 6-ethoxypurine is reduced is selected as a strain exhibiting more favorable growth compared with a non-modified strain, for example, the parent strain, when they are cultured in a medium containing 6-ethoxypurine, for example. That is, it can be said that, in the Bacillus bacterium of the present invention, sensitivity to 6-ethoxypurine is reduced, or the resistance to 6-ethoxypurine is improved, compared with the parent strain.

Examples of the aforementioned medium include, but are not limited to a minimal medium. Examples of the minimal medium include, for example, a medium having the following composition: 20 g/L of glucose, 5 g/L of ammonium chloride, 4 g/L of potassium dihydrogenphosphate, 0.01 g/L of ferrous sulfate, 0.01 g/L of manganese sulfate, 0.5 g/L of sodium citrate, pH 7.0.

In the present invention, the minimal medium may contain nutrients indispensable for the growth, if needed. For example, many of inosine producing strains are adenine auxotrophic, and therefore adenine is added to the medium at a concentration required for the growth. However, if the amount of adenine is too large, the growth inhibition effect of a certain kind of purine analogue may be reduced, and therefore the amount of adenine is preferably limited. Specifically, a concentration of about 0.1 g/L is preferred.

The selection of a target mutant strain or evaluation of growth inhibition by 6-ethoxypurine for the obtained mutant strain can be performed either in a liquid medium or on a solid medium. When a solid medium is used, for example, the mutant strain and the parent strain are each cultured in a liquid medium until a logarithmic growth phase or stationary phase is attained, then the culture broth is diluted with the medium, sodium chloride solution or the like, the obtained cell suspension is applied to a solid medium containing 6-ethoxypurine, and the mutant strain and the parent strain are cultured under the same conditions. The culture is usually performed at a temperature around the optimum growth temperature, for example, 34° C., for one to three days. Then, if the colonies of the mutant strain that emerge are larger than those of the parent strain, the growth of the mutant strain is more favorable than that of the parent strain, and growth inhibition by 6-ethoxypurine is reduced. The amount of ethoxypurine added to the medium is, for example, 1000 mg/L or more, preferably about 2000 mg/L.

Furthermore, when a liquid medium is used, cell suspensions of the mutant strain and the parent strain are cultured and diluted in the same manner as described above, and each inoculated into a liquid medium containing 6-ethoxypurine. The cells are cultured at a temperature around the optimum growth temperature, for example, 34° C., for several hours to one day, preferably about 6 hours. The amount of ethoxypurine added to the medium is, for example, 500 mg/L or more. If the mutant strain exhibits a higher optical density (OD) or turbidity of the medium either in, at least, a logarithmic growth phase or stationary phase, as compared with the parent strain, the growth is evaluated as favorable. Specifically, if the cells more quickly reach a logarithmic growth phase, or if the maximum value of the OD is higher, then growth is more favorable. The aforementioned logarithmic growth phase refers to a period in which the number of cells logarithmically increases on the growth curve. The stationary phase refers to a period after the logarithmic growth phase has passed and where cell division and growth have ceased, and the increase in cell number is no longer seen (Dictionary of Biochemistry, 3rd Edition, Tokyo Kagaku Dojin).

When a liquid medium is used, it may be more difficult to detect the difference in growth inhibition by 6-ethoxypurine as compared with when a solid medium is used. In the present invention, even if a difference in growth inhibition by 6-ethoxypurine cannot be detected using a liquid medium, a mutant strain is a strain which exhibits favorable growth as referred to in the present invention, so long as the mutant strain exhibits more favorable growth as compared with the parent strain on a solid medium.

Furthermore, the degree of growth inhibition by 6-ethoxypurine can also be evaluated by applying a suspension of the mutant strain to a solid medium containing 6-ethoxypurine and a solid medium not containing 6-ethoxypurine and comparing the size of the colonies that appear after culture under the same conditions as described above. For example, when a strain triply deficient in purR, purA and deoD derived from the Bacillus subtilis 168 Marburg strain was cultured at a 6-ethoxypurine content of 2000 mg/L in the medium, the relative growth degree calculated according to the aforementioned equation was about 40 to 60 with a culture time of 42 to 45 hours, or about 50 to 70 with a culture time of 48 to 66 hours, whereas the mutant strain obtained in the examples section showed a relative growth degree of about 80 to 100 with a culture time of 42 to 52 hours and the same content of 6-ethoxypurine. Therefore, by using the relative growth degree as an index, growth inhibition by 6-ethoxypurine can be evaluated without comparison with the parent strain. However, the growth inhibition by 6-ethoxypurine can also be evaluated by comparing the relative growth degrees of the parent strain and the mutant strain.

By culturing the Bacillus bacterium of the present invention obtained as described above in an appropriate medium, inosine can be produced and accumulated in the medium.

As for the medium used in the present invention, culture can be performed in a conventional manner using a typical medium containing a carbon source, nitrogen source and mineral salts as well as organic trace nutrients such as amino acids and vitamins, as required. Either a synthetic medium or a natural medium may be used. Any kind of carbon source and nitrogen source may be used so long as they can be utilized by the strain to be cultured.

As the carbon source, sugars such as glucose, glycerol, fructose, sucrose, maltose, mannose, galactose, starch hydrolysates and molasses can be used, and organic acids such as acetic acid and citric acid can also be used either alone or in combination with other carbon sources.

As the nitrogen source, ammonia, ammonium salts such as ammonium sulfate, ammonium carbonate, ammonium chloride, ammonium phosphate and ammonium acetate, nitric acid salts and so forth can be used.

As the organic trace nutrients, amino acids, vitamins, fatty acids, nucleic acids, those containing these substances such as peptone, casamino acid, yeast extract and soybean protein hydrolysate and so forth may be used. When an auxotrophic mutant strain that requires an amino acid or the like for its growth is used, it is necessary to supplement the required nutrient.

As the mineral salts, phosphoric acid salts, magnesium salts, calcium salts, ferrous salts, manganese salts and so forth can be used.

Although the culture condition may vary depending on the type of Bacillus bacterium to be used, Bacillus subtilis, for example, is cultured as aeration culture, while the fermentation temperature is controlled to be between 20 to 50° C., and pH to be between 4 to 9. When pH falls during the culture, the medium is neutralized with an alkali such as ammonia gas. Inosine is accumulated in the culture broth after 40 hours to 3 days of culture in a manner as described above.

After completion of the culture, inosine which has accumulated in the culture broth may be collected in a conventional manner. For example, inosine can be isolated by precipitation, ion exchange chromatography and so forth.

Furthermore, if the microorganism used for the present invention is made deficient in a gene coding for a nucleosidase or nucleotidase, a corresponding nucleoside or nucleotide can be accumulated. Furthermore, if inosine auxotrophy is imparted, a precursor or relevant substances involved in the biosynthesis pathway thereof can be accumulated.

Furthermore, by allowing purine nucleoside phosphorylase and/or phosphoribosyltransferase to act on inosine obtained by the method of the present invention, 5′-inosinic acid can be obtained.

### EXAMPLES

Hereinafter, the present invention will be explained more specifically with reference to the following non-limiting examples.

### Example 1

A recombinant which is derived from Bacillus subtilis (B. subtilis 168 Marburg strain, ATCC 6051) and which is deficient in the purine operon repressor gene (purR), succinyl-AMP synthase gene (purA) and purine nucleoside phosphorylase gene (deoD) was prepared as follows.

(1) Acquisition of Purine Operon Repressor Gene (purR) Deficient Strain

Based on information of a gene data bank (GenBank Accession No. Z99104), primers for PCR having the following nucleotide sequences, each of which was a 28-mer, were prepared.

| | |
|---|---|
| CTCAAGCTTGAAGTTGCGATGATCAAAA | (SEQ ID NO: 1) |
| CTCCTGCAGACATATTGTTGACGATAAT | (SEQ ID NO: 2) |

The chromosomal DNA of the B. subtilis 168 Marburg strain was used as a template together with the aforementioned primers to perform PCR (a cycle consisting of 94° C. for 30 second, 55° C. for 1 minute, and 72° C. for 1 minute was repeated for 30 cycles using Gene Amp PCR System Model 9600 (Perkin-Elmer)) to obtain an amplified fragment of about 0.9 kb of the purR gene region covering SD-ATG and the translation termination codon.

On the 5′-end of the aforementioned primers for PCR, a HindIII site and PstI site were designed, respectively. The PCR-amplified fragment was treated with HindIII and PstI and then ligated to the pHSG398 vector (Takara Shuzo) digested with the same restriction enzymes, which was replicable in E. coli, by using T4 DNA ligase, to obtain a pHSG398BSPR plasmid.

An internal sequence of about 0.3 kb in the purR structural gene existing between the EcoRV and HincII sites, each of which is unique in the purR structural gene, was removed from pHSG398BSPR plasmid by a treatment with EcoRV and HincII. Then, the spectinomycin resistance gene (1.2 kb) derived from Enterococcus faecalis and cloned in pDG1726 plasmid (which can be prepared from the Escherichia coli ECE101 strain commercially available from the Bacillus Genetic Stock Center (The Ohio State University, Department of Biochemistry (484 West Twelfth Avenue, Columbus, Ohio 43210 USA))) was excised as an EcoRV-HincII fragment and inserted between the aforementioned restriction enzyme sites of pHSG398BSPR.

The resulting plasmid pHSG398purR::spc was used to transform competent cells of the B. subtilis 168 Marburg strain prepared according to the method of Dubunau and Davidoff-Abelson (J. Mol. Biol., 56, 209 (1971)), and the colonies grown on an LB (10 g/L of trypton, 5 g/L of yeast extract, 10 g/L of NaCl, pH 7) agar plate containing 100 μg/ml of spectinomycin were selected. Chromosomal DNAs were prepared from these colonies, and a strain in which the purR gene on a chromosome was replaced with the purR gene of which internal sequence was replaced with the spectinomycin resistance gene (purR::spc) by double cross over recombination was identified by the aforementioned PCR method. One of the recombinant strains obtained as described above was designated as KMBS4.

(2) Acquisition of succinyl-AMP Synthase Gene (pura) Deficient Strain

Based on information of a gene data bank (GenBank Accession No. Z99104), primers for PCR having the following nucleotide sequences, each of which was a 29-mer, were prepared.

| | |
|---|---|
| CTCGTCGACAAAACGAATGGAAGCGAACG | (SEQ ID NO: 3) |
| CTCGCATGCAGACCAACTTATATGCGGCT | (SEQ ID NO: 4) |

The chromosomal DNA of the B. subtilis 168 Marburg strain was used as a template together with the aforementioned primers to perform PCR (a cycle consisting of 94° C. for 30 second, 55° C. for 1 minute, and 72° C. for 2 minute was repeated for 30 cycles using Gene Amp PCR System Model 9600 (Perkin-Elmer)) to obtain an amplified fragment of about 0.9 kb of the purA gene region covering SD-ATG and the translation termination codon.

On the 5′end of the aforementioned primers for PCR, an SalI site and SphI site were designed, respectively. The PCR-amplified fragment was treated with SalI and SphI and then ligated to the pSTV28 vector (Takara Shuzo) digested with the same restriction enzymes, which was replicable in E. coli, using T4 DNA ligase, to obtain a pSTV28BSPA plasmid.

An internal sequence of about 0.4 kb in the purA structural gene existing between the MluI site and BglII site, each of which is unique in the purA structural gene, was removed from pSTV28BSPA plasmid by a treatment with MluI and BglII, and the both ends of the DNA fragment containing the plasmid structure were blunt-ended using the Klenow fragment. To this DNA fragment, an erythromycin resistance gene (1.6 kb) derived from Staphylococcus aureus and cloned on the pDG646 plasmid (which can be prepared from the Escherichia coli ECE91 strain commercially available from the Bacillus Genetic Stock Center) was ligated using T4 DNA ligase, after it was excised from the pDG646 plasmid by a treatment with HindIII, and both ends were blunt-ended using the Klenow fragment.

The obtained plasmid pSTV28purA::erm was used to transform competent cells of the KMBS4 strain prepared according to the method of Dubunau and Davidoff-Abelson (J. Mol. Biol., 56, 209 (1971)), and the colonies grown on an LB agar plate containing 0.1 μg/ml of erythromycin and 125 μg/ml of lincomycin. Chromosomal DNAs were prepared from these colonies, and strains (purR::spc,purA::erm) in which the purA gene on a chromosome was replaced with the purA gene of which internal sequence was replaced with the erythromycin resistance gene (purA::erm) by double cross over recombination were identified by the aforementioned PCR method. The recombinant strains obtained as described above became adenine auxotrophic strains, and one of them was designated as KMBS13.

(3) Acquisition of Purine Nucleoside Phosphorylase Gene (deoD) Deficient Strain

(i) Cloning of the 5′-End Region of deoD

Based on information of a gene data bank (GenBank Accession No. Z99104), primers for PCR having the following nucleotide sequences, which were a 29-mer and 28-mer, respectively, were prepared.

| | |
|---|---|
| CTCGAATTCCAGCGGAATATTCTTTCCCG | (SEQ ID NO: 5) |
| CTCGGATCCCGGCAAAAGCACAGTATCC | (SEQ ID NO: 6) |

The chromosomal DNA of the B. subtilis 168 Marburg strain was used as a template together with the aforementioned primers to perform PCR (a cycle consisting of 94° C. for 30 second, 55° C. for 1 minute and 72° C. for 1 minute was repeated for 30 cycles using Gene Amp PCR System Model 9600 (Perkin-Elmer)) to obtain an amplified fragment containing about 310 bp upstream from the deoD gene translation initiation codon and about 60 bp downstream from the same.

On the 5′-end of the aforementioned primers for PCR, an EcoRI site and BamHI site were designed, respectively. The PCR-amplified fragment was treated with EcoRI and BamHI and then ligated to the pSTV28 vector (Takara Shuzo) digested with the same restriction enzymes by using T4 DNA ligase to obtain a pSTV28DON plasmid.

(ii) Cloning of the 3′-End Region of deoD

Based on information of a gene data bank (GenBank Accession No. Z99104), primers for PCR having the following nucleotide sequences, which were a 29-mer and 28-mer, respectively, were prepared.

| | |
|---|---|
| CTCAAGCTTATGGTTTCCAGACCATCGACT | (SEQ ID NO: 7) |
| CTCGGATCCCATGATATGATAGAAGTGG | (SEQ ID NO: 8) |

The chromosomal DNA of the B. subtilis 168 Marburg strain was used as a template together with the aforementioned primers to perform PCR (a cycle consisting of 94° C. for 30 second, 55° C. for 1 minute and 72° C. for 1 minute was repeated for 30 cycles using Gene Amp PCR System Model 9600 (Perkin-Elmer)) to obtain an amplified fragment containing about 40 bp upstream from the deoD gene translation termination codon and about 320 bp downstream from the same.

On the 5′-end sides of the aforementioned primers for PCR, a HindIII site and BamHI site were designed, respectively. The PCR-amplified fragment was treated with HindIII and BamHI and then ligated to the pSTV28DON plasmid digested with the same restriction enzymes using T4 DNA ligase to obtain a pSTV28DONC plasmid.

(iii) Insertion of Kanamycin Resistance Gene into deoD

Based on information of a gene data bank (GenBank Accession No. V01547), primers for PCR having the following nucleotide sequences, each of which was a 33-mer, were prepared.

| | |
|---|---|
| CTCGGATCCGAGGTGATAGGTAAGATTATACCG | (SEQ ID NO: 9) |
| CTCGGATCCGGCGCTCGGGACCCCTATCTAGCG | (SEQ ID NO: 10) |

The pDG783 plasmid containing the kanamycin resistance gene derived from Streptococcus faecalis (Bacillus Genetic Stock Center) was used as a template together with the aforementioned primers to perform PCR (a cycle consisting of 94° C. for 30 second, 55° C. for 1 minute and 72° C. for 1 minute was repeated for 30 cycles using Gene Amp PCR System Model 9600 (Perkin-Elmer)) to obtain an amplified fragment having about 1.5 kb of the kanamycin resistance gene region covering SD-ATG and the translation termination codon.

On each of the 5′-ends of the primers for PCR, a BamHI site was designed. The PCR-amplified fragment was treated with BamHI and inserted into the pSTV28DONC plasmid at the BamHI site, which was unique in the plasmid.

The resulting plasmid pSTV28deoD::kan was used to transform competent cells of the KMBS13 strain prepared according to the method of Dubunau and Davidoff-Abelson (J. Mol. Biol., 56, 209 (1971)), and the colonies grown on an LB agar plate containing 5 μg/ml of kanamycin. Chromosomal DNAs were prepared from these colonies, and strains in which the deoD gene on a chromosome was replaced with the deoD gene of which internal sequence was replaced with the kanamycin resistance gene (deoD::kan) by two times of recombination (purR::spc purA::erm deoD::kan) were identified by the aforementioned PCR method. One of the double recombinant strains obtained as described above was designated as KMBS 16.

### Example 2

### Selection of 6-ethoxypurine-resistant Strain

( [symbol] ) Screening of 6-Ethoxypurine Resistant Strain

The Bacillus subtilis recombinant strain KMBS16 deficient in the purine operon repressor gene (purR), succinyl-AMP synthase gene (purA) and purine nucleoside phosphorylase gene (deoD) was treated with 400 μg/ml of nitrosoguanidine (NTG) at 37° C. for 30 minutes. Then, the NTG-treated cell suspension was spread on a minimal medium (20 g/L of glucose, 5 g/L of ammonium chloride, 4 g/L of potassium dihydrogenphosphate, 0.01 g/L of ferrous sulfate, 0.01 g/L of manganese sulfate, 0.5 g/L of sodium citrate, 1.0 g/L of L-glutamic acid, 0.1 g/L of adenine, 50 mg/L of tryptophan, pH 7.0) plate containing 2000 mg/L of 6-ethoxypurine, and the cells were cultured at 37° C. for 24 hours. Among the colonies exhibiting favorable growth on the plate containing 2000 mg/L of 6-ethoxypurine, ten strains were selected and designated as EP1 to EP10 strains. The EP1 strain was given a private number of AJ13937. This strain was deposited at the independent administrative agency, National Institute of Advanced Industrial Science and Technology, International Patent Organism Depositary (Tsukuba Central 6, 1-1, Higashi 1-Chome, Tsukuba-shi, Ibaraki-ken, 305-8566, Japan) on Dec. 25, 2001 and received an accession number of FERM P-18665. Then, it was transferred to an international deposit under the provisions of the Budapest Treaty on Jan. 14, 2004, and received an accession number of FERM BP-08595.

When resistance of the ethoxypurine-resistant strain obtained as described above to various drugs conventionally used for the breeding of known inosine producing bacteria was investigated, it exhibited a tendency similar to that of the parent strain.

(2) Assay for degree of 6-ethoxypurine Resistance

Degree of 6-ethoxypurine resistance of the mutant strain obtained as described above was examined in detail. The parent strain and the mutant strain were adenine auxotrophic, and the drug used was a purine analogue. Therefore, the amount of adenine added to the minimal medium was limited to the required minimum level (2 mg/L in this example).

The 6-ethoxypurine-resistant strains (EP1 strain and EP2 strain) and the parent strain KMBS16 were each inoculated into the minimum liquid medium and cultured overnight at 34° C. The cells were separated from the culture broth by centrifugation, washed three times with the minimal medium and suspended again in the same medium. The suspension was appropriately diluted with the same medium and spread on a minimal medium plate and a minimal medium plate containing 2000 mg/L of 6-ethoxypurine, and the cells were cultured at 34° C. After 24 hours, minute colonies were confirmed for the EP1 strain and EP2 strain, whereas no colony was confirmed for the parent strain KMBS16. The culture was continued even thereafter, and diameters of several single colonies were measured after 48 hours to 66 hours. An average of colony diameters was calculated for each strain, and the relative growth degree was calculated according to the aforementioned equation.

The diameters of colonies measured as described above and relative growth degrees are shown in Tables 1 to 3. In the tables, the symbol “−” means that no colony could be confirmed, and the symbol “±” means that since the colonies were indefinite, the diameters could not be measured.

TABLE 1
Diameter of colony on minimal medium plate not containing
6-ethoxypurine

| | | Diameter of colony (mm) | | | | | |
|---|---|---|---|---|---|---|---|
| Strain | NO. | 28 hr | 42 hr | 45 hr | 48 hr | 52 hr | 66 hr |
| KMBS16 | 1 | ± | 1 | 1.2 | 1.5 | 1.5 | 2.0 |
| EP1 | 1 | ± | 1 | 1.2 | 1.5 | 1.5 | 2.0 |

TABLE 2
Diameter of colony on minimal medium plate containing
6-ethoxypurine (2000 μg/mL)

| | | Diameter of colony (mm) | | | | | |
|---|---|---|---|---|---|---|---|
| Strain | NO. | 28 hr | 42 hr | 45 hr | 48 hr | 52 hr. | 66 hr |
| KMBS16 | 1 | − | 0.50 | 0.70 | 1.00 | 1.00 | 1.25 |
| | 2 | − | 0.40 | 0.50 | 0.75 | 1.00 | 1.00 |
| EP1 | 1 | ± | 0.80 | 1.00 | 1.20 | 1.50 | 2.50 |
| | 2 | ± | 1.00 | 1.20 | 1.50 | 1.50 | 2.00 |
| EP2 | 1 | ± | 1.00 | 1.20 | 1.50 | 1.50 | 2.00 |
| | 2 | ± | 1.00 | 1.20 | 1.50 | 1.50 | 2.00 |

TABLE 3
Relative growth degree

| | | Relative growth degree (%) | | | | | |
|---|---|---|---|---|---|---|---|
| Strain | NO. | 0 hr | 42 hr | 45 hr | 48 hr | 52 hr | 66 hr |
| KMBS16 | 1 | 0 | 50.0 | 58.3 | 66.7 | 66.7 | 62.5 |
| | 2 | 0 | 40.0 | 41.7 | 50.0 | 66.7 | 50.0 |
| EP1 | 1 | 0 | 80.0 | 83.3 | 80.0 | 100.0 | |
| | 2 | 0 | 100.0 | | | | |
| EP2 | 1 | 0 | 100.0 | | | | |
| | 2 | 0 | 100.0 | | | | |

### Example 3

### Production of Purine Nucleic Acids by 6-ethoxypurine-resistant Strain

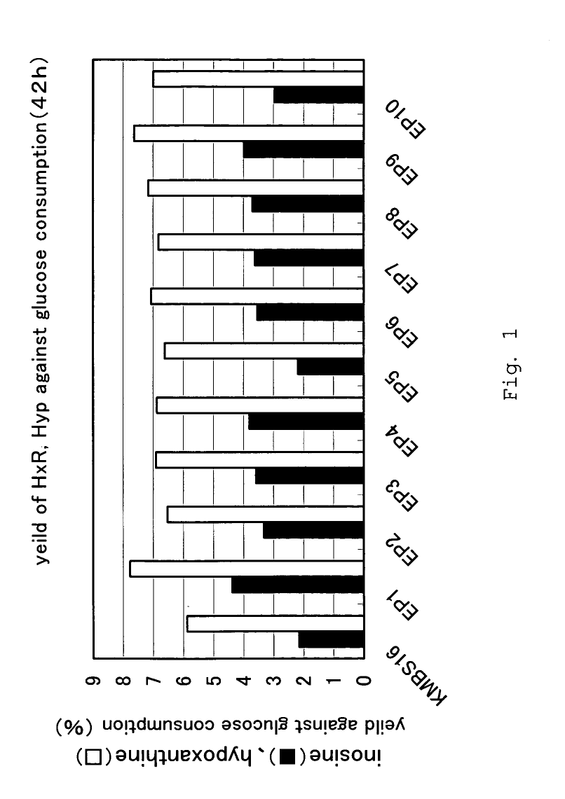

The 6-ethoxypurine-resistant strain (EP1 strain) and the parent strain KMBS16 were each uniformly applied on an LB medium plate and cultured overnight at 37° C. The cells of 1/6 of the plate were inoculated into 20 ml of fermentation medium in a 500 ml volume elemental flask, and after calcium carbonate was added to the medium at a concentration of 50 g/L, the cells were cultured at 37° C. with shaking. After the start of the culture, the medium was sampled in the time course, and the amounts of inosine and hypoxanthine contained in the culture broth were measured using known methods. All of the 6-ethoxypurine-resistant strains exhibited higher inosine and hypoxanthine accumulations compared with the parent strain KMBS16. As representative examples, the results measured after 42 hours are shown (FIG. 1).

[Composition of a fermentation medium]

| | |
|---|---|
| Glucose | 80 g/L |
| KH₂PO₄ | 1 g/L |
| NH₄Cl | 32 g/L |
| Mameno (T-N)* | 1.35 g/L |
| DL-Methionine | 0.3 g/L |
| L-Tryptophan | 0.02 g/L |
| Adenine | 0.1 g/L |
| MgSO₄ | 0.4 g/L |
| FeSO₄ | 0.01 g/L |
| MnSO₄ | 0.01 g/L |
| GD113 | 0.01 ml/L |
| (adjusted to pH 7.0 with KOH) | |
| Calcium carbonate | 50 g/L |

*Soybean protein hydrolysates
**Anti-foam-reagent

What is claimed is:

1. A Bacillus bacterium which is modified so that growth inhibition by 6-ethoxypurine is reduced and has inosine-producing ability.

2. The Bacillus bacterium according to claim 1, which is a mutant strain derived from a parent strain belonging to the genus Bacillus and shows favorable growth as compared with the parent strain when cultured in a medium containing 6-ethoxypurine.

3. The Bacillus bacterium according to claim 2, wherein the medium has an ethoxypurine content of 2000 mg/L.

4. The Bacillus bacterium according to claim 1, wherein the medium is a solid medium.

5. The Bacillus bacterium according to claim 1, wherein when the bacterium is cultured by applying a suspension of the bacterium to a solid medium containing 6-ethoxypurine and a solid medium not containing 6-ethoxypurine, the bacterium shows a relative growth degree of 80 or more, which is defined by the following equation:
Relative growth degree (%)=[colony diameter (mm) observed in the medium containing 6-ethoxypurine]/[colony diameter (mm) observed in the medium not containing 6-ethoxypurine]×100.

6. The Bacillus bacterium according to claim 5, wherein the solid medium containing 6-ethoxypurine has a 6-ethoxypurine content of 2000 mg/L.

7. The Bacillus bacterium according to claim 6, wherein the solid medium is a minimal medium.

8. The Bacillus bacterium according to claim 1, which is deficient in one or more genes negatively acting on the biosynthesis of inosine or involved in degradation of inosine and selected from a purine operon repressor gene, succinyl-AMP synthase gene and purine nucleoside phosphorylase gene.

9. A method for producing a Bacillus bacterium having improved inosine-producing ability, which comprises selecting strains showing favorable growth in a medium containing 6-ethoxypurine from a population of Bacillus bacteria, and selecting a strain showing high inosine-producing ability from the obtained strains.

10. The method according to claim 9, wherein the population of Bacillus bacteria is obtained by subjecting a parent strain belonging to the genus Bacillus to a mutagenesis treatment.
